(19) United States
(12) Patent Application Publication
(10) Pub. No.: US 2002/0076831 A1
(43) Pub. Date: Jun. 20, 2002

(54) Test piece, analysis method using the test piece, and analysis system used for the method

(76) Inventor: Taizo Akimoto, Kaisei-machi (JP)

Correspondence Address: SUGHRUE, MION, ZINN, MACPEAK & SEAS, PLLC, 2100 Pennsylvania Avenue, N. W., Washington, DC, 20037-3202, US

(21) Appl. No.: 09/749,752

(22) Filed: Dec. 28, 2000

(30) Foreign Application Priority Data
Dec. 28, 1999 (JP) 372925/1999
Dec. 28, 1999 (JP) 372926/1999

(51) Int. Cl. G01N 33/543

(52) U.S. Cl. 436/518; 422/58; 436/56

(57) ABSTRACT

A test piece having plural types of probes, an analysis method using the test piece and an analysis system used for the method effectively prevent incorrect association between one set of information concerning detected positions of the probes to which a target substance has bound and the other set of information concerning types and positions of all probes arranged and fixed on the test piece. As management information itself peculiar to the test piece or ID information indicating the content of the management information has been attached to a predetermined location on the test piece, the information concerning the detected positions of the probes to which the target substance has bound may be associated correctly with corresponding management information by detecting the management information or the ID information attached on the test piece concurrently with the positions of the probes to which the target substance has bound.

### BACKGROUND OF THE INVENTION

1. Field of the Invention

The present invention relates to a test piece such as a micro-array, a macro-array or a DNA chip, an analysis method using the test piece, and an analysis system used for the method. More specifically, the present invention relates to: a test piece having a base carrying plural types of probes (e.g., organic molecules) arranged and fixed thereon; an analysis method for identifying positions of those probes on the test piece to which a target substance (e.g., organic molecules) marked with a marker (e.g., radioactive isotope or fluorescent dye) has bound; and an analysis system used for the method.

2. Description of the Related Art

A method for analyzing gene expressions using a micro-array is described in detail in the paper titled “Gene Expression Analysis Using the Micro-Array” (Experimental Medical Science Series Vol. 17; p.1-5; Yodosha Shuppann, Inc. (January, 1999)).

Such methods of gene expression analysis using the micro-array, a macro-array, a DNA chip, etc., have recently come into wide use. A test piece as shown in FIG. 8 is used in those methods. The test piece has a base plate 40 (e.g., a membrane, a glass plate, a slide glass or a silicon base plate) on a surface of which plural kinds of organic molecules (frequently used are, e.g., c-DNA, oligo-DNA, other types of DNA, PNA and EST) are arranged and fixed in matrix as probes using a spotting device etc. Such a test piece may be referred to by either of a variety of names (e.g., a macro-array, a micro-array or a DNA chip) according to the type or size of the base plate 40, the number of array-points, size of each array-point, the types of the substances used as the probes, the type of the target substance, etc.

The target substance contains organic molecules (e.g., molecules of c-DNA, genom-DNA, m-RNA, other types of RNA, dNTP or PNA) marked with radioactive isotope, fluorescent dye, etc.

In the analysis, the marked target substance hybridizes selectively with some of the probes fixed in a matrix.

Those probes containing the organic molecules capable of hybridizing (or combining) with the organic molecules contained in the target substance hybridize (or combine) with the target substance, and thereby the marker such as the radioactive isotope or the fluorescent dye is fixed to the several probes on the base. On the other hand, the marker would not be fixed to those probes not containing the organic molecules capable of hybridization. Double circles in FIG. 8 schematically indicate the positions on the base where the hybridized probes reside, i.e., the positions where the marker is fixed. Although each of the array-points arranged in matrix is clearly separated from others for explanatory purposes in FIG. 8, each of the array-points can hardly be distinguished visually from others in practice as they are very small and arranged in close proximity to each other at a high density.

The positions of the hybridized probes on the test piece can be identified by detecting the positions where the marker is fixed. Subsequently, the types of the probes can be identified based on the identified positions thereof.

As described above, the process of identifying the types of the hybridized probes always includes the step of comparing two sets of information, i.e., one set of information concerning the detected positions of the marker and the other set of information concerning the type and the position of each probe arranged and fixed on the test piece.

However, an examiner has heretofore been required to manually input to a computer the set of information concerning the type and the position of each probe to identify the types of the hybridized probes based on the set of information concerning the detected positions of the marker, because it has been typical that the set of information concerning the type and the position of each probe is kept stored in the spotting device etc. used for preparing the test piece. Hence, when conducting experiments on a plurality of test pieces each carrying several types of probes arranged thereon in a different pattern, it has sometimes occurred that the examiner inputs the set of information concerning the wrong test piece or that the experiment is carried out on the wrong test piece. In those cases, correct combinations cannot be obtained between the information concerning the detected positions of the probes to which the marker has fixed and the information concerning the type and the position of each probe.

### SUMMARY OF THE INVENTION

The object of the present invention is to provide: a test piece effective in preventing incorrect association between two sets of information, i.e., one set of information concerning the detected positions of the probes to which the target substance has bound and the other set of information concerning the type and the position of each probe arranged and fixed on the test piece; an analysis method using the test piece; and an analysis system used for the method.

The field of use of the test piece according to the present invention is not necessarily limited to the field of gene analysis, e.g., in the field of gene expressions analysis, base sequence identification, variation analysis or polymorphism analysis. The test piece of the present invention can instead be used for analysis of any target substance as long as the target substance is capable of selectively bonding to the probes, which are arranged and fixed in matrix on a base, through any kind of reaction.

A test piece according to the present invention comprises: plural types of probes arranged and fixed thereon, to some of which a target substance marked with a marker binds selectively; and a pattern of ID information peculiar to the test piece attached to a predetermined location on the test piece.

The pattern of the ID information is preferably printed on the test piece using a marker the same as or similar to the one used for marking the target substance.

The test piece according to the present invention may take any form, e.g., the form of a micro-array, a macro-array or a DNA chip, as long as it remains capable of carrying plural types of probes arranged thereon.

The substances used as the probes may be of any kind, e.g., c-DNA, oligo-DNA, other types of DNA, PNA or EST, as long as it is suitable for use in array analysis. That is to say, the substance arranged and fixed as a probe on the test piece of the present invention is not limited to organic molecules but may be of any kind as long as it remains capable of being combined selectively with the target substance through any kind of reaction.

Typical examples of the target substance include organic molecules such as the molecules of c-DNA, genom-DNA, m-RNA, total-RNA, other types of RNA, dNTP or PNA. However, any appropriate substance other than those organic molecules listed above may also be used.

The “predetermined location” to which the ID information is attached may be any location on the test piece other than those spots where the probes reside.

The term “ID information” refers to the information for distinguishing each test piece from others. The information is satisfactory at least for identifying the type and the position of each probe arranged and fixed on that test piece. The ID information indicates the content of the management information peculiar to the test piece, such as the type of the base of the test piece, the date of preparing the test piece, a serial number, a lot number, the types of the substances used as the probes and the positions of the probes. Herein, it is preferable to have the ID information as an encoded form of the management information in order to minimize the space on the test piece required for attaching the ID information. The ID information may additionally indicate the contents of the management information concerning the target substance as well.

Typical examples of the marker include radioactive isotope and fluorescent dye. However, any other appropriate marker may also be selcted.

The pattern of the ID information may be attached on the test piece using a spotting device, an ink jet printer, etc.

An analysis method for analyzing a target substance according to the present invention comprises the steps of: causing the target substance marked with a marker to bond selectively to some of plural types of probes arranged and fixed on a test piece by bringing the target substance marked with the marker into contact with the test piece; attaching ID information peculiar to the test piece at a predetermined location on the test piece; obtaining information concerning positions of the probes to which the target substance has bound; and detecting the ID information.

The possible forms of bonding between the target substance and the probes include hybridization, in which a pair of complementary base sequences form a stable double strand, and any other specific form of bonding. The possible types of reactions through which the target substance bonds to the probes include, for example, several types of affinity.

The information concerning the positions of the probes to which the target substance has bound can be obtained by detecting the positions of the marker fixed to the probes, i.e., the positions where the target substance marked with the marker binds to the probes on the test piece.

In the method according to the present invention, the ID information is preferably printed on the test piece using a marker the same as or similar to the one used for marking the target substance.

In this context, “a marker similar to the one used for marking the target substance” refers to a marker which is different from the marker used for marking the target substance but which emits radiation similar to the radiation emitted by the marker used for marking the target substance, in the case where a radioactive isotope is selected as the marker used for marking the target substance, or refers to a marker which is different from the marker used for marking the target substance but which emits fluorescence having a wavelength range similar to the fluorescence emitted by the marker used for marking the target substance when exposed to stimulating light having a wavelength range similar to that for the marker used for marking the target substance, in the case where a fluorescent dye is selected as the marker used for marking the target substance.

The step of attaching the ID information may be carried out at any point in time before the step of obtaining the information concerning the positions of the probes to which the target substance has bound. For example, the ID information may be attached upon arrangement of the probes on the test piece or may be attached after the step of causing the target substance to bond to the probes.

According to the analysis method of the present invention in which the ID information peculiar to the test piece is attached to the test piece, incorrect association between one set of information concerning the detected positions of the probes to which the target substance has bound and the other set of information concerning the type and the position of each probe arranged and fixed on the test piece may be prevented effectively, by detecting the ID information, which has been correctly associated with the management information including the type and the position of each probe, concurrently with the information concerning the positions of the probes to which the target substance has bound.

In this respect, the ID information and the information concerning the positions of the probes to which the target substance has bound may be detected simultaneously requiring no additional step, i.e., the step of detecting the ID information may be incorporated into the step of obtaining the information concerning the positions of the probes to which the target substance has bound, when printing the ID information using the marker the same as or similar to the marker used for marking the target substance.

According to a first aspect of the present invention, there is provided an analysis system for analyzing a target substance by causing the target substance marked with a marker to bind selectively to some of plural types of probes arranged and fixed on a test piece and detecting positions of the probes to which the target substance has bound comprising: means for attaching management information peculiar to the test piece to a predetermined location on the test piece; means for obtaining information concerning the positions of the probes to which the target substance has bound; means for detecting the management information attached to the test piece; and means for storing the management information in association with the information concerning the positions of the probes to which the target substance has bound.

It is desirable that the analysis system according to the first aspect of the present invention further comprises means for searching through the means for storing referring to specified management information to find the information concerning the positions of the probes to which the target substance has bound associated with the specified management information.

In the analysis system according to the first aspect of the present invention, the management information is preferably printed on the test piece using a marker the same as or similar to the one used for marking the target substance.

According to a second aspect of the present invention, there is provided an analysis system for analyzing a target substance by causing the target substance marked with a marker to bind selectively to some of plural types of probes arranged and fixed on a test piece and detecting positions of the probes to which the target substance has bound comprising: means for attaching encoded management information to a predetermined location on the test piece as ID information peculiar to the test piece; means for obtaining information concerning the positions of the probes to which the target substance has bound; means for detecting the ID information attached to the test piece; first storing means for storing the management information in association with the ID information; means for decoding the detected ID information by searching through the first storing means referring to the detected ID information to find the management information associated with the detected ID information; and second storing means for storing the management information in association with the information concerning the positions of the probes to which the target substance has bound.

It is desirable that the analysis system according to the second aspect of the present invention further comprises means for searching through the second storing means referring to specified management information to find the information concerning the positions of the probes to which the target substance has bound associated with the specified management information.

In the analysis system according to the second aspect of the present invention, the ID information is preferably printed on the test piece using a marker the same as or similar to the one used for marking the target substance.

The term “management information” refers to a set of information concerning the characteristics of a test piece. The management information is satisfactory at least for identifying the type and the position of each probe arranged and fixed on that test piece. For example, the management information may include information such as the type of the base of the test piece, the date of preparing the test piece, a serial number, a lot number, the types of the substances used as the probes and the positions of the probes. The management information may also include the information concerning the target substance.

The meaning of the phrase “to associate the management information with the ID information” in this context is to relate the certain management information to certain ID information so that the certain management information may be derived based the ID information related thereto. For example, an ID number may be provided to each test piece as the ID information thereof so that the management information of a corresponding test piece may be derived referring to the ID number provided thereto.

With the analysis system according to the first aspect of the present invention in which the management information peculiar to the test piece is attached to the test piece, incorrect association between one set of information concerning the detected positions of the probes to which the target substance has bound and the other set of information concerning the type and the position of each probe arranged and fixed on the test piece may be prevented effectively, as the means for storing stores the management information in association with the information concerning the positions of the probes to which the target substance has bound.

In addition, with the analysis system according to the first aspect of the present invention, an examiner can check the results of the analysis (i.e., correct combination of the information concerning the positions of the probes to which the target substance has bound and the management information including the information concerning the type and the position of each probe) by specifying the desired management information after the analysis, because the means for storing may be searched through referring to the specified management information to find the corresponding information concerning the positions of the probes to which the target substance has bound. Accordingly, the examiner may identify the types of the probes to which the target substance has bound.

Moreover, the management information and the information concerning the positions of the probes to which the target substance has bound may be detected simultaneously requiring no additional apparatus, i.e., the function of the means for detecting the management information may be incorporated into the means for obtaining information concerning the positions of the probes to which the target substance has bound, when printing the management information using the marker the same as or similar to the marker used for marking the target substance.

With the analysis system according to the second aspect of the present invention in which the ID information (i.e., the encoded management information) peculiar to the test piece is attached to the test piece, incorrect association between one set of information concerning the detected positions of the probes to which the target substance has bound and the other set of information concerning the type and the position of each probe arranged and fixed on the test piece may be prevented effectively, as the second storing means stores the management information in association with the information concerning the positions of the probes to which the target substance has bound.

In addition, with the analysis system according to the second aspect of the present invention, an examiner can check the results of the analysis (i.e., correct combination of the information concerning the positions of the probes to which the target substance has bound and the management information including the information concerning the type and the position of each probe) by specifying the desired management information after the analysis, because the second storing means may be searched through referring to the specified management information to find the corresponding information concerning the positions of the probes to which the target substance has bound. Accordingly, the examiner may identify the types of the probes to which the target substance has bound.

Moreover, the ID information and the information concerning the positions of the probes to which the target substance has bound may be detected simultaneously requiring no additional apparatus, i.e., the function of the means for detecting the ID information may be incorporated into the means for obtaining information concerning the positions of the probes to which the target substance has bound, when printing the ID information using the marker the same as or similar to the marker used for marking the target substance.

### BRIEF DESCRIPTION OF THE DRAWINGS

FIG. 1 is a perspective view of a test piece,

FIG. 2 is a perspective view of one embodiment of the test piece according to the present invention, schematically showing how probes are arranged and fixed in matrix on the surface thereof,

FIG. 3A and FIG. 3B show exemplary patterns to be attached to the test piece representing content of management information,

FIG. 4 is a perspective view of the test piece of FIG. 2, schematically illustrating the process of hybridization,

FIG. 5 is a perspective view of the test piece of FIG. 2 after hybridization,

FIG. 6 is a perspective view of the test piece of FIG. 2, schematically illustrating the process of exposing an stimulable phosphor sheet superposed on the test piece to the radiation emitted from the radioactive isotope on the test piece,

FIG. 7 is a perspective view of the stimulable phosphor sheet being exposed to a stimulation laser beam for readout,

FIG. 8 is a perspective view of a conventional micro-array,

FIG. 9 schematically shows the configuration of an

analysis system according to one embodiment of the present invention, and

FIG. 10 is a flowchart illustrating a series of processes carried out by the analysis system shown in FIG. 9.

### DESCRIPTION OF THE PREFERRED EMBODIMENTS

Now, an analysis method for analyzing organic molecules using a micro-array according to the present invention will be described with reference to the accompanying drawings.

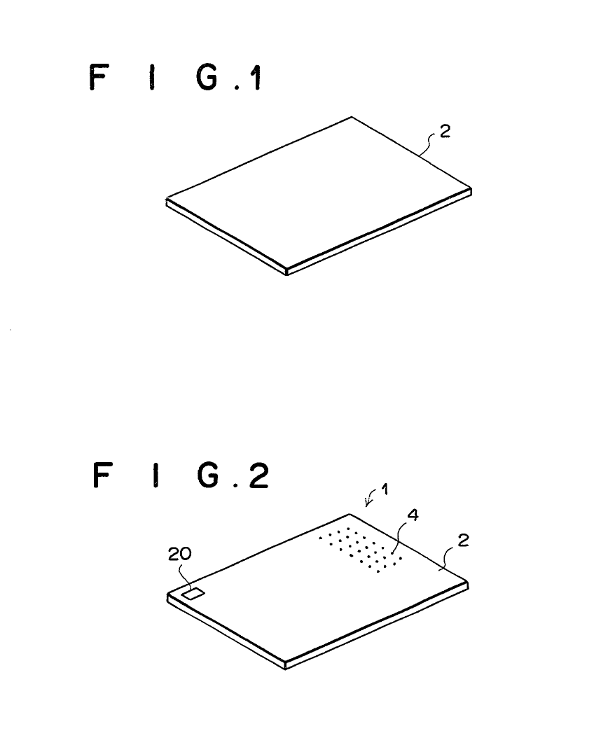

First of all, plural types of organic molecules (plural types of cDNAs in this example) are arranged in matrix on a membrane (or a base) using a spotter. That is to say, plural types of cDNAs 4 are arranged in matrix on the membrane 2 to form a test piece 1 as shown in FIG. 1. Each dot on the membrane 2, at which one of the plural types of cDNAs is alloted, can be regarded as a probe.

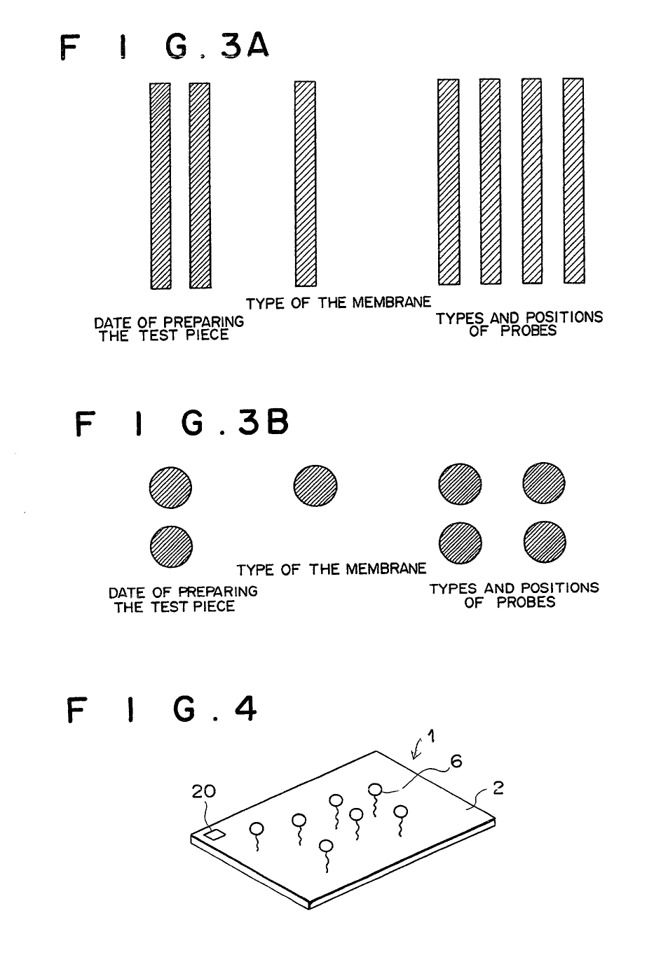

Next, ID information (i.e., encoded management information) peculiar to the test piece 1 is attached to a predetermined location 20 (see FIG. 2) on the test piece 1. Specifically in the present embodiment, a certain radioactive isotope (e.g., ¹⁴C, ³²P or ³³P) is disposed on the predetermined location 20 in a pattern representing the content of the management information using the same spotter as the one used for arranging cDNAs on the membrane 2. If there was a long time interval scheduled between preparation of the test piece 1 and hybridization thereof, it would be preferable to use ¹⁴C which has a long half life. The radioactive isotope used herein for printing the ID information to the test piece 1 may be the same radioactive isotope as the one used for marking a target substance on a later stage, or may be a radioactive isotope which is different from the one used for marking the target substance but which emits radiation similar to the radiation emitted by the radioactive isotope used for marking the target substance. FIG. 3A and FIG. 3B show exemplary forms of the ID information, i.e., exemplary patterns of the radioactive isotope to be disposed on the predetermined location 20 of the test piece 1. As shown in FIG. 3A and FIG. 3B, the management information peculiar to the test piece 1 is encoded in to the ID information and then attached to the surface of the test piece 1. Specifically, the management information may include information such as the date of preparing the test piece 1, a serial number, the type of the membrane, the types of the substances used as the probes and the positions of the probes. Any appropriate conventional method may be used for encoding the management information into the ID information.

The management information may be encoded into ID information in a bar-code pattern as shown in FIG. 3A or into ID information in a dotted pattern as shown in FIG. 3B. In FIG. 3A and FIG. 3B, the pattern of the ID information is constituted of three portions each representing the date of preparing the test piece 1, the type of the membrane 2, or the types of the substances used as the probes and the positions of the probes, respectively. However, the entire contents represented by those three portions may instead be represented by a single portion. The encoding of the management information into the ID information enables the test piece 1 to accommodate the content of the management information in a small area thereon.

After arranging the probes on the surface of the membrane 2 following the process explained above, preparation of the test piece 1 is completed by projecting ultraviolet radiation onto the membrane 2 to thereby fix the probes to the membrane 2.

In the next step, a cDNA marked with a radioactive isotope is synthesized through reverse transcription using as the template poly(A)RNA prepared from the RNA extracted from the cells to be analyzed. This marked cDNA is the target substance in the present embodiment.

If there were known information concerning the target substance, it would be desirable to add such information after encoding thereof to the ID information attached to the test piece 1.

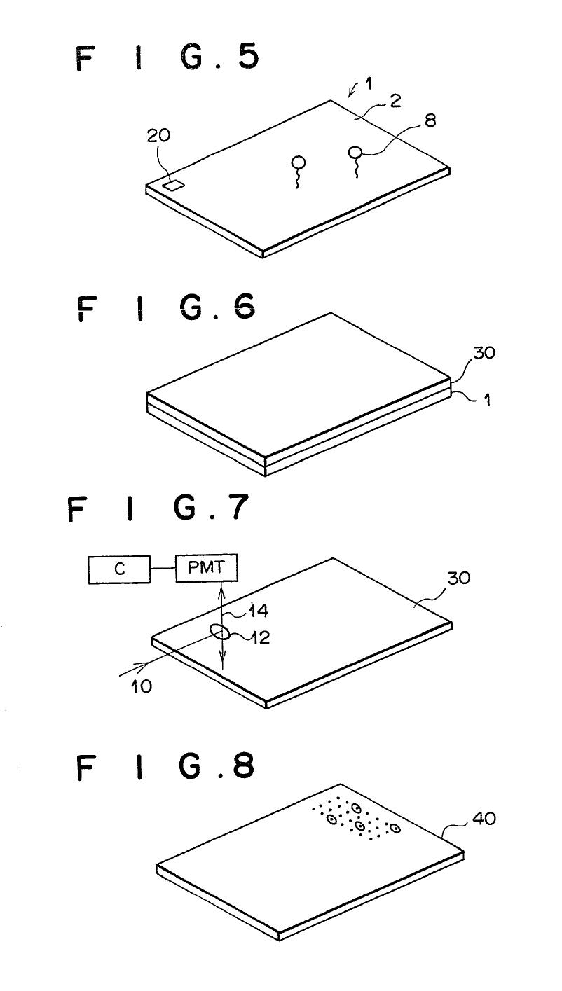

The test piece 1 is then soaked in a prepared solution containing the target substance, whereby the probes of certain types hybridize with the target substance (see FIG. 4). Subsequently, the excessive targets 6 which have not been hybridized are washed away from the surface of the test piece 1 leaving on the surface only those targets 8 hybridized with the probes (see FIG. 5). The hybridized targets 8 have been marked with the radioactive isotope.

In the next step, the positions of those probes hybridized with targets 8 are detected by superposing an stimulable phosphor sheet 30 fixedly on the test piece 1 (see FIG. 6) and leaving the test piece 1 overlaid with the stimulable phosphor sheet 30 in a dark place to expose the stimulable phosphor sheet 30 to the radiation emitted from the radioactive isotope fixed on the test piece 1. Herein, visible light is projected in advance over the entire surface of the stimulable phosphor sheet 30 to erase unnecessary information stored thereon before superposing the stimulable phosphor sheet 30 on the test piece 1. Accumulated on the stimulable phosphor sheet 30 after a predetermined period of exposure are the radiation energy emitted from the radioactive isotope marking the targets 8 left on the test piece 1 and the radiation energy emitted from the radioactive isotope disposed at the location 20 in the pattern of the ID information.

The term “stimulable phosphor sheet” refers to a phosphor sheet which absorbs and accumulates radiation energy when exposed to radiation and which emits the accumulated radiation energy as light in the manner of stimulated emission when subsequently irradiated with stimulating light such as a laser beam having a certain wavelength range. A typical example of a well-known stimulable phosphor sheet, which is known also as a radiation inverting panel, utilizes a phosphor which exhibits stimulated emission, and is constituted of a base sheet overlaid with a layer of a material composed of a binder and stimulable phosphor particles such as BaFX particles (X represents a halogen atom) dispersed in the binder at a high density.

In the next step, the stimulable phosphor sheet 30 is detached from the test piece 1 to measure the radiation energy accumulated thereon. In FIG. 7, which illustrates the way of measuring the radiation energy accumulated on the stimulable phosphor sheet 30, the entire surface of the stimulable phosphor sheet 30 is sweeped with a stimulation laser beam 10 reflected by a mirror 12, which is either of a half mirror or a dichroic mirror. A spot on the stimulable phosphor sheet 30 irradiated with the stimulation laser beam 10 emits a beam of stimulated emission 14. The beam of stimulated emission 14 then passes through the mirror 12 and is detected by a photomultiplier (PMT) . Accordingly, the positions of the radioactive isotope on the test piece 1 can be determined, because detected by the PMT is the stimulated emission emitted from those spots on the stimulable phosphor sheet 30 corresponding to the positions of the radioactive isotope on the test piece 1. Those spots on the stimulable phosphor sheet 30 include two kinds of spots, i.e., the spots accumulating the radiation energy emitted from the radioactive isotope used as the marker of the targets 8 hybridized selectively with some of the probes and the spot accumulating the radiation energy emitted from the radioactive isotope disposed at the location 20 in the pattern of the ID information peculiar to the test piece 1. The stimulated emission detected by the PMT is then converted to an electric signal. The electric signal is sent to a computer C and the information concerning the positions of the radioactive isotope on the test piece 1 is stored in the computer C.

The information concerning the positions of the radioactive isotope stored in the computer C contains the ID information, which is peculiar to the test piece 1 and is attached to the predetermined location 20 in FIG. 5, and the ID information can be decoded into specific management information (i.e., the date of preparing the test piece 1, the type of the membrane, the types of the substances used as the probes, the positions of the probes, etc.).

As the first step of analyzing the information stored in the computer C in the present embodiment, the information concerning the positions of the radioactive isotope disposed in a pattern on the location 20 is analyzed to decode the ID information indicated by the pattern into the corresponding management information (e.g., the date of preparing the test piece 1, the type of the membrane, the types of the substances used as the probes and the positions of the probes). The management information is then associated with the information concerning the positions of the hybridized probes. Therefore, an examiner may now recognize which cDNA has or has not hybridized with the RNA extracted from the cells to be analyzed, by inputting to the computer C a specific set of management information to derive the corresponding information concerning the positions of the hybridized probes (i.e., cDNAs), and having the computer C compare the derived information with the information concerning the type and position of each probe (i.e., cDNA) contained in the management information.

In the analysis method according to the above embodiment of the present invention, in summary, the stimulable phosphor sheet 30 is capable of storing the ID information, i.e., the encoded form of the management information peculiar to the test piece 1 (e.g., the date of preparing the test piece 1, the type of the membrane, the types of the substances used as the probes and the positions of the probes), as the ID information has been attached to the test piece 1 using the radioactive isotope the same as the one used as the marker. Accordingly, the ID information stored on the stimulable phosphor sheet 30 may be detected concurrently with the information concerning the positions of the hybridized probes stored on the same stimulable phosphor sheet.

In addition, incorrect association between the information concerning the position and type of each probe and the information concerning the detected positions of the hybridized probes would be prevented effectively, even if experiments on many types of probes were required and thus there were many test pieces to be examined, as correct association would be achieved referring to the ID information attached to the test piece 1.

Moreover, in the above embodiment, the ID information and the information concerning the positions of the hybridized probes can be detected simultaneously requiring no additional step, as the radioactive isotope the same as or similar to the radioactive isotope used as the marker is used for printing the ID information to the test piece 1.

In addition, an ink jet printer may be used in place of the spotter used in the above embodiment for attaching the ID information to the membrane 2. In that case, the ID information is printed on the surface of the membrane 2 using the radioactive isotope as ink.

Although the ID information was attached to the test piece 1 before hybridization in the above embodiment, the ID information may instead be attached after hybridization. In that case, the radioactive isotope disposed on the test piece 1 in the pattern of the ID information does not require the fixation process. This is because there is no possibility of the radioactive isotope on the test piece 1 peel off after hybridization, while the radioactive isotope may peel off during hybridization or the subsequent process of washing away the excessive target substance.

In addition, fluorescent dye (e.g., Cy5 or Cy3) may be used in place of the radioactive isotope used in the above embodiment as the marker to mark the target substance. In that case, the PMT can detect the direct information from the test piece without the use of the stimulable phosphor sheet by projecting stimulating light capable of stimulating the fluorescent dye directly onto the test piece 1.

Although the radioactive isotope the same as or similar to the radioactive isotope used as the marker is used for attaching the ID information to the test piece 1 in the above embodiment, the ID information may instead be attached through normal printing or a similar method, e.g., the method of forming an embossed pattern or an engraved pattern on the membrane 2 as the ID information representing the content of the management information.

Now, an analysis system for detecting positions of those probes on a test piece hybridized with marked targets and identifying the types of the hybridized probes will be described as another embodiment of the present invention with reference to the accompanying drawings.

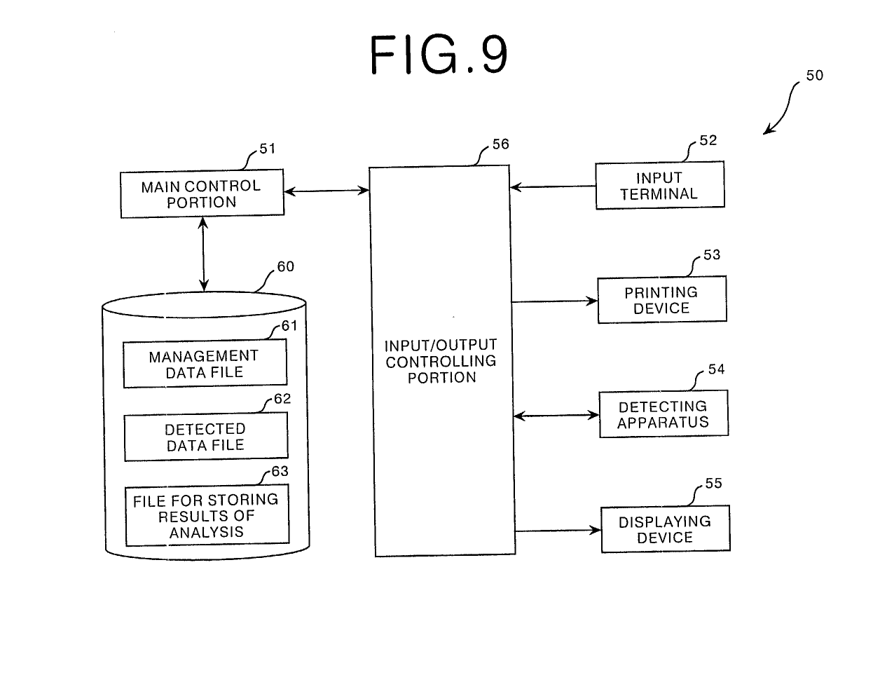

This analysis system detects the positions of the targets 8 in FIG. 5, i.e., the targets hybridized with certain types of probes on the test piece 1 as described above. The configuration of the analysis system will now be described in reference to FIG. 9.

An analysis system 50 according to the present embodiment is constituted of a main control portion 51 suitably programmed for controlling general action of the analysis system 50, a memory 60 which is connected to the main control portion 51 and which is capable of storing plural kinds of data, an input terminal 52 (e.g., a keyboard) connected to the main control portion 51 via an input/output controlling portion 56, a printing device 53 for printing on a test piece 1 ID information (i.e., encoded management information) peculiar to the test piece 1, a detecting apparatus 54 for detecting positions of radioactive isotope fixed to the test piece 1, and a displaying device 55 (e.g., a display) for displaying thereon, for example, information concerning the positions of the radioactive isotope detected by the detecting apparatus 54.

The main control portion 51 has an internal memory for storing a controlling program such as an OS (operating system), a program for encoding the management information concerning the test piece 1 inputted to the main control portion 51 into the ID information, a program for extracting the ID information out of entire information concerning the detected positions of the radioactive isotope on the test piece 1, a program for controlling the detecting apparatus 54 and the printing device 53, a program for controlling the content to be displayed on the displaying device 55, and required data of any kind. The above programs stored in the internal memory of the main control portion 51 can be written using known programming techniques.

The memory 60, which may be of any suitable form (e.g., a hard disk, a flexible disk or an optical disk), stores a management data file, a detected data file 62 and a file 63 for storing results of analysis. The management data file 61 stores the management information concerning the test piece 1 in association with the corresponding ID information (i.e., the encoded form of the management information) attached to the test piece 1. The management information may include, for example, the date of preparing the test piece 1, a serial number, the type of a membrane 2 of the test piece 1, the types of the substances used as the probes and the positions of the probes. The management information stored in the management data file 61 may also include information concerning the target substance, e.g., the type of the target substance and conditions of hybridization, if such information was known in advance of a hybridization process. Instead, such information concerning the target substance may be added to the management data file 61 after the hybridization process. The test piece 1 is distinguished from other test pieces by the ID information (i.e., an encoded form of the management information) attached thereto. In addition, the management information concerning the test piece 1, i.e., the types of probes thereon etc., can be identified by searching through the management data file 61 referring to the ID information attached to the test piece 1. The detection data file 62 stores the information concerning the positions of the radioactive isotope detected by the detecting apparatus 54. The file 63 for storing the results of the analysis stores the information concerning the detected positions of the radioactive isotope in association with a correct set of the management information concerning the test piece 1.

Used as the printing device 53 in the present embodiment is a spotter which is also used for arranging the probes on the membrane 2.

In the present embodiment, the positions of the radioactive isotope (e.g., ¹⁴C, ³²P or ³³P) on the test piece 1 are transcribed onto an stimulable phosphor sheet for detection. Thus, the detecting apparatus 54 can be selected from known apparatuses capable of measuring the radiation energy accumulated on the stimulable phosphor sheet (e.g., the apparatus disclosed in Japanese Unexamined Patent Publication 10(1998)-3134).

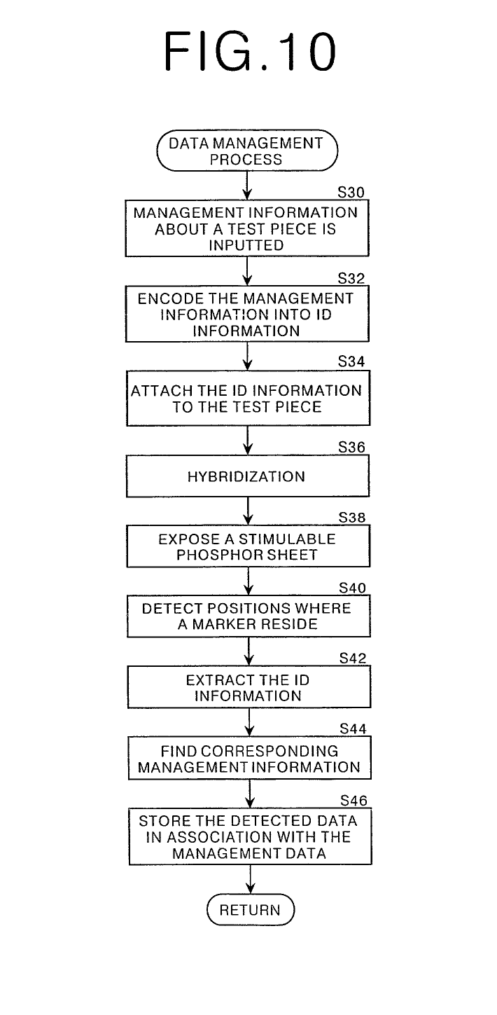

Now, a series of processes carried out by the analysis system 50 according to the present embodiment will be described in reference with FIG. 10.

Initially, the management information concerning the test piece 1 is inputted to the analysis system 50 at the input terminal 52 (S.30 in FIG. 10). Although the management information in the present embodiment only includes the date of preparing the test piece 1, the type of the membrane 2 of the test piece 1, the types of the substances used as the probes and the positions of the probes, it may also include additional information concerning the test piece such as a serial number and a lot number. The inputted management information is transferred through the input/output controlling portion 56 and the main control portion 51 to the management data file 61 for storage on one hand, and is encoded at the main control portion 51 on the other hand (S.32). The encoded management information is then attached as the ID information to the predetermined location 20 on the test piece 1 (see FIG. 5) using the radioactive isotope the same as the one used as the marker (S.34). That is to say, in the present embodiment, the radioactive isotope is disposed using the printing device 53 in a certain pattern representing the content of the management information concerning the predetermined location 20 on the test piece 1. FIG. 3A and FIG. 3B show exemplary forms of the ID information, i.e., exemplary patterns of the ID information to be disposed on the predetermined location 20 on the test piece 1.

In the next step, the target substance marked with the radioactive isotope is hybridized with some of the probes on the test piece 1 (S.36). Subsequently, the positions of those probes hybridized with the targets 8 are detected by superposing the stimulable phosphor sheet 30 fixedly on the test piece 1 (see FIG. 6) and leaving the test piece 1 overlaid with the stimulable phosphor sheet 30 in a dark place to expose the stimulable phosphor sheet 30 to the radiation emitted from the radioactive isotope fixed on the test piece 1 (S.38). Herein, visible light is projected in advance over the entire surface of the stimulable phosphor sheet 30 to erase unnecessary information stored thereon before superposing the stimulable phosphor sheet 30 on the test piece 1. Accumulated on the stimulable phosphor sheet 30 after a predetermined period of exposure are the radiation energy emitted from the radioactive isotope marking the targets 8 left on the test piece 1 and the radiation energy emitted from the radioactive isotope isposed at the location 20 in the pattern of the ID information.

Subsequently, the stimulable phosphor sheet 30 is detached from the test piece 1 and the radiation energy accumulated on the stimulable phosphor sheet is measured by the detecting apparatus 54 (S.40). The detecting apparatus 54 acts in the way already described in reference to FIG. 7, in which a photomultiplier (PMT) in the detecting apparatus 54 detects stimulated emission emitted from the stimulable phosphor sheet 30. The stimulated emission detected by the PMT is then converted to an electrical signal. The electrical signal is transferred through the input/output controlling portion 56 and the main control portion 51 to the detection data file 62, and the information concerning the detected positions of the radioactive isotope is stored in the detection data file 62.

In the next step of the present invention, the ID information (i.e., the encoded management information) attached to the location 20 on the test piece 1 is extracted from the entire information concerning the positions of the radioactive isotope stored in the detection data file 62 (S.42). The management data file 61 is then searched through referring to the extracted ID information to find a set of the management information associated with the ID information (S.44). Subsequently, the information concerning the detected positions of the hybridized probes is stored in the file 63 (i.e., the file for storing results of analysis) in association with the correct set of the management information concerning the test piece 1 (S.46).

The file 63 after the series of processes shown in FIG. 10 therefore holds two sets of information in association, i.e., the management information concerning the test piece 1 and the information concerning the detected positions of the hybridized probes on the test piece 1. Accordingly, the examiner can now obtain a desired set of the information concerning the detected positions of the hybridized probes referring to the management information associated therewith. In addition, once the series of processes shown in FIG. 10 are completed, the examiner can recognize which probe has or has not hybridized with the RNA extracted from the cells to be analyzed, by comparing the information concerning the detected positions of the hybridized probes with information concerning the type and position of each probe contained in the management information.

In the analysis system according to the above embodiment of the present invention, in summary, the stimulable phosphor sheet 30 is capable of storing the ID information peculiar to the test piece 1, i.e., the encoded form of the management information of the test piece 1 (e.g., the date of preparing the test piece 1, the type of the membrane, the types of the substances used as the probes and the positions of the probes), as the ID information has been printed on the test piece 1 by the printing device 53 using the radioactive isotope the same as the one used as the marker. Accordingly, the ID information stored on the stimulable phosphor sheet 30 may be detected concurrently with the information concerning the positions of the hybridized probes stored on the same stimulable phosphor sheet 30.

In addition, incorrect association between the management information and the information concerning the positions of the hybridized probes would be prevented effectively by referring to the ID information attached to each test piece, even if experiments on many types of probes were required and thus there were many test pieces to be examined, as the correct combinations of the management information and the ID information have been stored in the management data file 61 within the memory 60.

Moreover, in the above embodiment, the ID information and the information concerning the positions of the hybridized probes can be detected simultaneously requiring no additional apparatus, as the radioactive isotope the same as or similar to the radioactive isotope used for marking the target substance is used for printing the ID information to the test piece 1.

Although the ID information was attached to the test piece 1 (as shown in FIG. 2) before hybridization in the above embodiment, the ID information may instead be attached after hybridization. In addition, the information concerning the target substance may be added to the management data file 61 after the hybridization process.

Moreover, fluorescent dye (e.g., Cy5 or Cy3) may be used in place of the radioactive isotope used in the above embodiment as the marker to mark the target substance. In that case, the PMT can detect the direct information from the test piece without the use of the stimulable phosphor sheet by projecting stimulating light capable of stimulating the fluorescent dye directly onto the test piece 1.

Although the radioactive isotope the same as or similar to the radioactive isotope used as the marker is used for attaching the ID information to the test piece 1 in the above embodiment, the ID information may instead be attached through normal printing or a similar method, e.g., the method of forming an embossed pattern or an engraved pattern on the membrane 2 as the ID information representing the content of the management information.

Although the ID information, i.e., the encoded management information, is attached to the test piece 1 in the above embodiment, the management information itself may instead be attached directly to the test piece 1. The management information may be written in characters on the test piece 1. In that case, the detecting device 54 must be capable of character recognition to read the management information written on the test piece 1. In the case where the management information is attached directly to the test piece 1, there would be no management data file 61 stored in the memory 60 and the information concerning the positions of the hybridized probes would be stored in the data file 63 in association with the management information read directly from the test piece 1.

In addition, all of the contents of Japanese Patent Application Nos. 11(1999)-372925 and 11(1999)-372926 are incorporated into this specification by reference.

What is claimed is:

1. A test piece comprising
plural types of probes arranged and fixed thereon, to some of which a target substance marked with a marker binds selectively, and
a pattern of ID information peculiar to the test piece attached to a predetermined location on the test piece.

2. A test piece according to claim 1, wherein the pattern of the ID information is printed on the test piece using a marker the same as or similar to the marker used for marking the target substance.

3. A test piece according to claim 1 or 2, wherein the pattern of the ID information is attached on the test piece using a spotting device or an ink jet printer.

4. An analysis method for analyzing a target substance comprising the steps of
causing the target substance marked with a marker to bond selectively to some of plural types of probes arranged and fixed on a test piece by bringing the target substance marked with the marker into contact with the test piece,
attaching ID information peculiar to the test piece at a predetermined location on the test piece,
obtaining information concerning positions of the probes to which the target substance has bound, and
detecting the ID information.

5. An analysis method according to claim 4, wherein the ID information is printed on the test piece using a marker the same as or similar to the marker used for marking the target substance.

6. An analysis system for analyzing a target substance by causing the target substance marked with a marker to bind selectively to some of plural types of probes arranged and fixed on a test piece and detecting positions of the probes to which the target substance has bound comprising
means for attaching management information peculiar to the test piece to a predetermined location on the test piece,
means for obtaining information concerning the positions of the probes to which the target substance has bound,
means for detecting the management information attached to the test piece, and
means for storing the management information in association with the information concerning the positions of the probes to which the target substance has bound.

7. An analysis system according to claim 6, further comprising means for searching through the means for storing referring to specified management information to find the information concerning the positions of the probes to which the target substance has bound associated with the specified management information.

8. An analysis system according to claim 6 or 7, wherein the management information is printed on the test piece using a marker the same as or similar to the marker used for marking the target substance.

9. An analysis system for analyzing a target substance by causing the target substance marked with a marker to bind selectively to some of plural types of probes arranged and fixed on a test piece and detecting positions of the probes to which the target substance has bound comprising
means for attaching encoded management information to a predetermined location on the test piece as ID information peculiar to the test piece,
means for obtaining information concerning the positions of the probes to which the target substance has bound,
means for detecting the ID information attached to the test piece,
first storing means for storing the management information in association with the ID information,
means for decoding the detected ID information by searching through the first storing means referring to the detected ID information to find the management information associated with the detected ID information, and
second storing means for storing the management information in association with the information concerning the positions of the probes to which the target substance has bound.

10. An analysis system according to claim 9, further comprising means for searching through the second storing means referring to specified management information to find the information concerning the positions of the probes to which the target substance has bound associated with the specified management information.

11. An analysis system according to claim 9 or 10, wherein the ID information is printed on the test piece using a marker the same as or similar to the marker used for marking the target substance.
